(19) United States
(12) Patent Application Publication
(10) Pub. No.: US 2024/0351991 A1
(43) Pub. Date: Oct. 24, 2024

(54) PROCESS FOR THE PREPARATION OF AN OPTICALLY ACTIVE ISOXAZOLINE COMPOUND

(71) Applicant: SYNGENTA CROP PROTECTION AG, Basel (CH)

(72) Inventors: Denis GRIBKOV, Münchwilen (CH); Harry John MILNER, Münchwilen (CH)

(73) Assignee: SYNGENTA CROP PROTECTION AG, Basel (CH)

(21) Appl. No.: 18/687,372

(22) PCT Filed: Aug. 26, 2022
(86) PCT No.: PCT/EP2022/073855

§ 371 Date: Feb. 28, 2024

(30) Foreign Application Priority Data
Aug. 30, 2021 (EP) 21193759.4

(51) Int. Cl. C07D 261/04

(52) U.S. Cl. CPC C07D 261/04

(57) ABSTRACT

The present invention relates to a process for the preparation of a compound of formula (I) or an enriched composition comprising a compound of formula (I), by reacting a compound of formula (II), with hydroxylamine or its salt, a base, a chiral catalyst, and an organic solvent, wherein said base is an anion exchange resin.

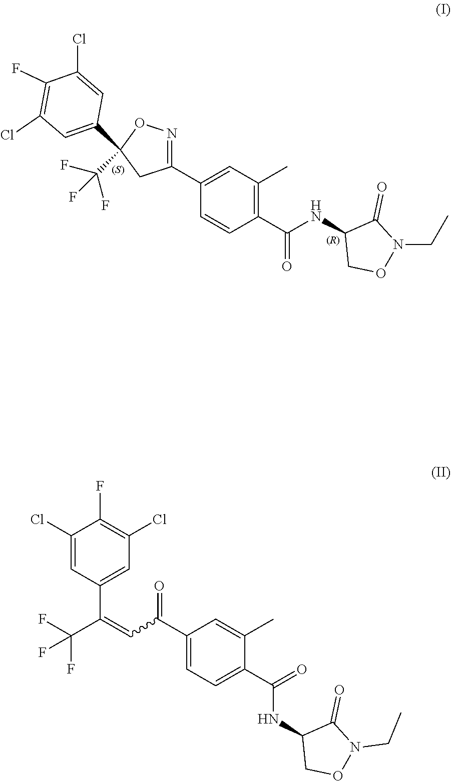

The present invention relates to a process for the preparation of an optically active isoxazoline compound of formula I, and to a process for the preparation of an enriched composition comprising an optically active isoxazoline compound of formula I, the optically active isoxazoline compound of formula I being useful as pesticide.

Processes for the preparation of optically active isoxazoline compounds are described, for example, in WO2016/023787. Optically active isoxazoline compounds with cycloserine substituent show two stereocentres which configuration is important for the biological activity of the compounds.

The reaction describes in WO2016/023787 gives cycloserine substituted isoxazolines with high stereoselectivity and low racemization. However, the presence of several isomers can affect the isolation process and the yield of the desired isomer.

Therefore, there is still a need to improve the enantioselectivity of the desired optically active product, especially for large scale production.

The aim of the present invention is to overcome the problems of the prior art techniques by proposing a process for the preparation of an optically active isoxazoline compound, especially with cycloserine substituent, which improves the enantioselectivity of the desired isomer, while guaranteeing a good chemical yield.

To this end, an object of the present invention is to provide a process for the preparation of a compound of formula I or an enriched composition comprising a compound of formula I

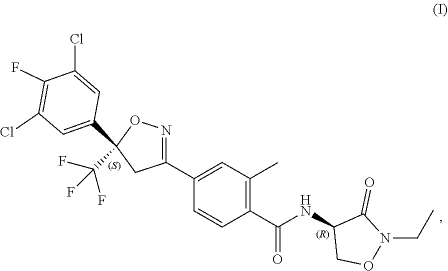

by reacting a compound of formula II

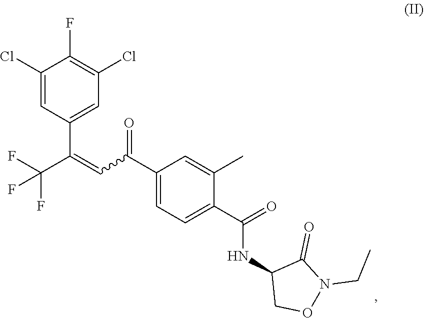 [SMILES: CCN1OC[C@@H](NC(=O)c2ccc(C(=O)C=C(c3cc(Cl)c(F)c(Cl)c3)C(F)(F)F)cc2C)C1=O]

with hydroxylamine or its salt, a base, a chiral catalyst, and an organic solvent, wherein said base is an anion exchange resin.

Thanks to said process, all the above problems have been overcome. More particularly, the present invention provides an increased enantioselectivity of the desired isomer, while guaranteeing a good chemical yield (especially greater than 90%). It can also be advantageously used for large scale production.

The process according to the present invention relates to the preparation of the isomer (5S,4R) of the compound of formula I, which is 4-[(5S)-5-(3,5-dichloro-4-fluoro-phenyl)-5-(trifluoromethyl)-4H-isoxazol-3-yl]-N-[(4R)-2-ethyl-3-oxo-isoxazolidin-4-yl]-2-methyl-benzamide. The process according to the present invention can also relate to the preparation of an enriched composition comprising the compound of formula I (5S,4R) and at least one of the isomers of the compound of formula I selected among isomer (5S,4S), isomer (5R,4R), isomer (5R,4S), and any combinations thereof.

In the present invention, the isomer (5S,4S) is 4-[(5S)-5-(3,5-dichloro-4-fluoro-phenyl)-5-(trifluoromethyl)-4H-isoxazol-3-yl]-N-[(4S)-2-ethyl-3-oxo-isoxazolidin-4-yl]-2-methyl-benzamide; the isomer (5R,4R) is 4-[(5R)-5-(3,5-dichloro-4-fluoro-phenyl)-5-(trifluoromethyl)-4H-isoxazol-3-yl]-N-[(4R)-2-ethyl-3-oxo-isoxazolidin-4-yl]-2-methyl-benzamide; and the isomer (5R,4S) is 4-[(5R)-5-(3,5-dichloro-4-fluoro-phenyl)-5-(trifluoromethyl)-4H-isoxazol-3-yl]-N-[(4S)-2-ethyl-3-oxo-isoxazolidin-4-yl]-2-methyl-benzamide.

The enriched composition can comprise a molar proportion of the isomer (5S,4R) greater than 50%, e.g. at least 55%, 60%, 65%, 70%, 75%, 80%, 85%, 90%, 95%, 96%, 97%, 98% or 99%, over the total amount of the isomers (5S,4R), (5S,4S), (5R,4R) and (5R,4S).

The base according to the present invention is an anion exchange resin, and more particularly a strong base anion (SBA) exchange resin.

An anion exchange resin can generally comprise a positively charged matrix and exchangeable anions.

More preferably, the anion exchange resin can be an OH anion exchange resin. In this case, the exchangeable anions are hydroxide anions (OH⁻). It is also possible to obtain an OH anion exchange resin from other types of anion exchange resins. For example, a chloride (Cl⁻ ) anion exchange resin can be used to obtain an OH anion exchange resin by rinsing said chloride anion exchange resin with an aqueous solution of NaOH until the active chloride anion sites are exchanged by hydroxide anions. Excess of aqueous solution of NaOH can be finally removed by rinsing the resin with demineralized water.

The matrix of the anion exchange resin can be a gel matrix or a microporous matrix, crosslinked or not. This type of matrix can comprise a polystyrenic matrix or a polyacrylic matrix. For example, the matrix can comprise a copolymer of styrene-divinylbenzene.

The anion exchange resin may be provided in any form, more particularly in any solid form. For example, the anion exchange resin may be provided as beads, and more particularly as spherical beads. The beads may have a size across their largest dimension (particle diameter) of from about 0.3 mm to about 1.2 mm, and more preferably from about 0.5 mm to about 0.8 mm.

In a particular embodiment, the anion exchange resin can comprise a functional group, such as quaternary ammonium functional group. More particularly, the anion exchange resin can be aminated with trimethylamine, and can comprise the trimethyl ammonium functional group. The anion exchange resin has typically an exchange capacity, well-known as Total Exchange Capacity on a water-wet basis, of the anion form, which can be of at least 0.50 equivalent per liter (eq/L), and preferably of at least 0.80 eq/L. In the process according to the present invention, the amount of exchangeable anions (based on the exchange capacity of the anion exchange resin) can be from 0.01 to 10 molar equivalents, preferably from 0.05 to 5 molar equivalents, preferably from 0.05 to 1.5 molar equivalents, and more preferably from 0.05 to 0.2 molar equivalents.

In the present invention, the expression “molar equivalents” is based on the number of moles (mol) of the compound of formula II.

According to the present invention, the anion exchange resin can be for example the AmberLite™ resin supplied by Dupont, such as AmberLite™ IRN78 OH Ion Exchange Resin, AmberLite™ HPR4800 OH Ion Exchange Resin (also well-known as Dowex Marathon™ A OH Ion Exchange Resin), or AmberLite™ A26 OH Polymeric Catalyst.

The organic solvent according to the present invention can comprise any suitable organic solvent well-known in the art. For example, the organic solvent can be selected among dichloromethane, 1,2-dichloroethane, toluene, chlorobenzene, chloroform, tert-butyl methyl ether, iso-propanol, ethanol, tetrahydrofuran, 2-methyltetrahydrofuran, acetonitrile, propionitrile, 2-methylpropionitrile, butyronitrile, and any combinations thereof. The preferred organic solvent can be selected among acetonitrile, iso-propanol, propionitrile, tetrahydrofuran, and any combinations thereof.

In the process according to the present invention, the amount of the organic solvent can be from 1 to 200 molar equivalents, and preferably from 10 to 100 molar equivalents.

The reaction may be carried out in the presence of water, or in other words the process can further comprise water. The weight ratio of organic solvent: water, and more preferably of the preferred organic solvent: water, can be from 200:1 to 1:1, and preferably from 100:1 to 5:1. The amount of water in said weight ratio refers to the total amount of water in the process, which can for example come from an aqueous hydroxylamine solution, a wet resin, and/or by adding water directly in the process.

The process according to the present invention comprises hydroxylamine or its salt, and preferably hydroxylamine. The term “hydroxylamine” means the free hydroxylamine of formula H₂NOH, and the hydroxylamine salts can be for example hydroxylammonium chloride. When OH anion exchange resin and hydroxylamine (H₂NOH) are used during the reaction, the hydroxylamine can get in contact with the OH anions of the OH anion exchange resin, so that OH anions can deprotonate the hydroxylamine and form water. The exchangeable anions can be in this case hydroxylamine anion (NH₂O⁻). In the process according to the present invention, the amount of hydroxylamine or its salts can be from 0.5 to 10 molar equivalents, preferably from 0.5 to 5 molar equivalents, and more preferably from 1.0 to 1.5 molar equivalents.

The chiral catalyst according to the present invention is more particularly a catalyst comprising at least one chiral moiety, and preferably at least two chiral moieties.

The chiral catalyst can comprise any suitable chiral catalyst well-known in the art.

In a first example, the chiral catalyst can be the compounds of formula III described on page 2 in WO2016/023787 (incorporated by reference), preferably the dimeric chiral catalyst of formula III described on page 4 in WO2016/023787, and more preferably the compound R-(6-methoxy-4-quinolyl)-[(2S)-1-[[2,3,5,6-tetrafluoro-4-[[(2S)-2-[(R)-hydroxy-(6-methoxy-4-quinolyl)methyl]-5-vinyl-quinuclidin-1-ium-1-yl]methyl]phenyl]methyl]-5-vinyl-quinuclidin-1-ium-2-yl]methanol dibromide (TFBBQ) with the following CAS number: 1879067-61-4 described as compound of formula XVII on page 8 in WO2016/023787. In WO2016/023787 pages 7-8, said compound of formula XVII can be prepared from the compound of formula XV with a suitable halogenating reagent such as SOBr₂, POBr₃, PBr₃, HBr, NaBr/H₂SO₄, or any combinations thereof; in a suitable solvent such as acetic acid, toluene, xylene, chlorobenzene, dichloromethane, heptane, ethyl acetate, dichloromethane, tetrahydrofuran, 2-methyltetrahydrofuran, 1,4-dioxane, dimethylformamide, N-methyl pyrrolidone, water, or any combinations thereof; to yield the compound of formula XVI. Then the compound of formula XVI can react with the compound of formula X in the presence of a suitable organic solvent such as toluene, acetonitrile, acetone, methanol, ethanol, 1-pentanol, tetrahydrofuran, 2-methyltetrahydrofuran, 1,4-dioxane, dimethyl formamide, N-methyl pyrrolidone, anisole, water, or any combinations thereof, to yield the compound of formula XVII.

In a second example, the chiral catalyst can be the compounds of formula 2 to 12 as chiral phase transfer catalysts, described in US2014350261A1 (incorporated by reference).

In a third example, the chiral catalyst can be the compounds of formula III described in WO2020/094434 (incorporated by reference)) or described in WO2021/197880 (incorporated by reference).

In the process according to the present invention, the amount of the chiral catalyst can be from 0.001 to 1.0 molar equivalents, and preferably from 0.01 to 0.5 molar equivalents.

The preparation of the compound of formula II is based on a dehydration reaction, said reaction being well-known in the art. The compound of formula II can be prepared, for example according to WO 2011/067272, in particular shown in Scheme 3 on pages 18-19. More particularly, the compound of formula II can be prepared by reacting a compound of formula III

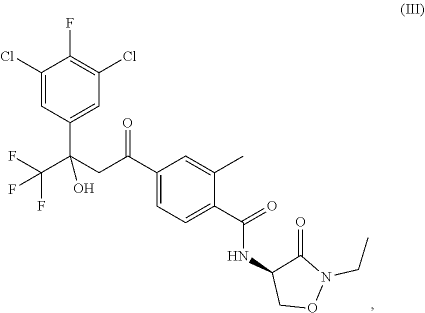 [SMILES: CCN1OC[C@@H](NC(=O)c2ccc(C(=O)CC(O)(c3cc(Cl)c(F)c(Cl)c3)C(F)(F)F)cc2C)C1=O]

in an organic solvent such as hexane, heptane, methycyclohexane, toluene, xylene, chlorobenzene, o-dichlorobenzene, dichloroemethane, dioxane, tetrahydrofuran, 2-methyltetrahydrofuran, cyclopentylethylether, anisole, acetonitrile, propionitrile, butyronitrile, benzonitrile, or any combinations thereof; with a base such as triethylamine, tri-n-butylamine, pyridine, or any combinations thereof; a dehydration agent such as phosgene, thionyl chloride, acetic anhydride, acetyl chloride, methanesulfonyl chloride, oxalyl chloride, methyl chloroformate, ethyl chloroformate, or any combinations thereof; and a catalyst such as aminopyridine catalyst which can be for example 4-dimethylaminopyridine or 4-pyrrolidinopyridine. Said mixture can be stirred in a reactor for about 10 minutes to 96 hours, and preferably about 1 to 20 hour(s), usually at 0 to 150° C., preferably at 0 to 20° C., and more preferably at 0 to 10° C. The compound of formula II can be isolated with work-up conditions well-known in the art, in separating the base, the dehydration agent, the catalyst or its respective reaction products from the compound of formula II.

In a first embodiment, the compound of formula II according to the present invention can comprise the E-configuration compound of formula II, and optionally the Z-configuration compound of formula II. More particularly, the compound of formula II can comprise a E/Z ratio from 90:10 to 100:0, preferably from 95:5 to 100:0, and more preferably from 99:1 to 100:0.

In a second embodiment, the compound of formula II according to the present invention can comprise a R/S ratio from 50:50 to 100:0, preferably from 90:10 to 100:0, and more preferably from 95:5 to 100:0.

In a third embodiment, the compound of formula II according to the present invention can comprise the first embodiment and the second embodiment.

The process according to the present invention can be carried out at a temperature ranging from −78° C. to 80° C., preferably from −20° C. to +20° C., and preferably from −20° C. to 0° C. The reaction time is usually from 30 minutes to 48 hours, and preferably from 1 to 4 hours. The process can be carried out in dosing at least one of the reactants selected among the hydroxylamine or its salt; the anion exchange resin; the chiral catalyst; the compound of formula II; and any combinations thereof. Dosing a reactant is well-known in the art and refers to the addition of several amounts of a compound over a predetermined period of time.

In a particular embodiment, the process according to the present invention can further comprise, after yielding the compound of formula I, a separation step to remove the anion exchange resin.

This separation step can be carried out by techniques well-known in the art such as for example by decantation, centrifugation or filtration (e.g. in using a centrifuge, a filternutsche, a candle filter, or a pocket filter). Before and/or after the separation of the resin, the pH of the reaction mixture can be adjusted and, if necessary, the reaction mixture heated to dissolve the compound of formula I. The reaction mixture can be adjusted to a pH of from 4 to 8, and preferably of from 5 to 6, using, for example, an acid such as hydrochloric acid (HCl). To dissolve the compound of formula I, the reaction mixture can be heated up to a temperature from 15 to 50° C.

The preparation of the compound of formula III as described beforehand, is based on a aldol reaction, said reaction being well-known in the art. More particularly, the compound of formula III can be prepared by reacting an aromatic ketone compound of formula IV

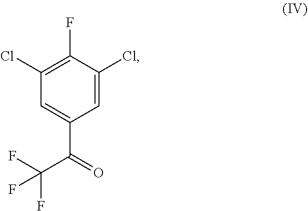 [SMILES: O=C(c1cc(Cl)c(F)c(Cl)c1)C(F)(F)F]

with a substituted acetophenone compound of formula V

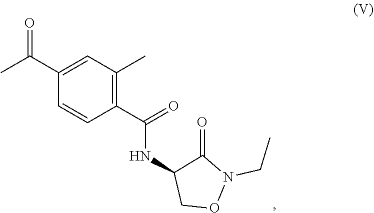 [SMILES: CCN1OC[C@@H](NC(=O)c2ccc(C(C)=O)cc2C)C1=O]

in the presence of a base such as triethylamine, trimethylamine, diethylamine, tert butylamine, pyridine, 1,8-diaza (5,4,0)-7-bicycloundecene, potassium carbonate, or any combination thereof; with or without a solvent. The solvent can be for example selected among toluene, xylene, chlorobenzene, dichlorobenzene, anisole, dimethoxybenzene, dioxane, tetrahydrofuran, 2-methyltetrahydrofuran, dimethylcarbonate, ethyl acetate, methoxyethyl acetate, and any combinations thereof. The equilibrium of the reaction can be shifted towards the compound of formula III by adjusting the amount of solvent in such a way that the the reaction is run as concentrated as possible with sufficient mixing. The mixture can be a homogenous solution or can be a slurry. Said mixture can be stirred in a reactor for about 1 to 150 hours, and preferably about 1 to 96 hour(s), usually at 0 to 150° C., preferably at 20 to 60° C., and more preferably at 30 to 50° C. The compound of formula III can be isolated or can be used without further work-up as such, to generate the compound of formula II.

Another object of the present invention relates to the use of an anionic exchange resin as defined in the description, in a process for preparing an isoxazoline group from the cyclisation of a chalcone group.

An isoxazoline group can be defined as a five-membered heterocyclic chemical compound, comprising one atom each of oxygen and nitrogen which are located adjacent to one another. A chalcone group can be defined as an α,β-unsaturated ketone such as a trans-1,3-diaryl-2-propen-1-one, comprising two aromatic rings attached by α,β-unsaturated carbonyl system with variety of substituents.

In a preferred embodiment, this other object can relate to the use of an anionic exchange resin as defined in the present invention, in a process for preparing the compound of formula I from a compound of formula II.

Another object of the present invention relates to a compound of formula I or an enriched composition comprising a compound of formula I, obtained by a process according to the present invention. The compound of formula I and the enriched composition comprising a compound of formula I are as defined respectively in the present invention.

The following non-limiting examples demonstrate the improved behaviour associated with a process according to the present invention.

Said examples provide a process according to the present invention (Example 1) and a comparative example (Example 2).

The components used in the below Examples 1 and 2 are detailed as follows:
compound of formula II is a mixture of the four isomers E,R; E,S; Z,R and Z,S of formula II with the following ratios: 98.6% (E,R); 1.3% (E,S); 0.1% (Z,R) and 0.0% (Z,S);
base 1 is an anion exchange resin (solid form), commercialized by Dupont under the name AmberLite™ IRN78 OH Ion Exchange Resin;
base 2 is sodium hydroxide 10% aqueous solution;
hydroxylamine is hydroxylamine 50% aqueous solution;
chiral catalyst is TFBBQ (CAS-No. 1879067-61-4); and
organic solvent is acetonitrile.

### Preparation of Example 1 (Ex. 1)

In a 20 L double jacketed reactor, 9083 g of organic solvent was charged at room temperature. Stirrer was started with reactor jacket set to −18° C. 288 g of hydroxylamine was charged, followed by 228 g of deionized water. 203 g of base 1 was added, followed by 106 g of chiral catalyst. When internal temperature has reached −18° C., dosing is started of overall 1999 g of compound of formula II, in 12 portions of 167 g over 1 hour. The highest internal temperature reached during addition was around −14° C. After the last portion of compound of formula II has been added, the reaction mass was continued to stir for 2 hours. The reaction mixture was sampled to confirm full conversion. After full conversion, 108 ml HCl 32% aqueous solution was added over 5 min to obtain a pH ˜5.0. The reaction mixture was warmed to 45° C. and base 1 was filtered off. A sample was taken for analysis (determination of chemical yield and isomer ratio).

### Preparation of Example 2 (Ex. 2)

In a 20 L double jacketed reactor, 9083g of organic solvent was charged at room temperature. followed by the addition of 263g of base 2 under stirring. The mixture was cooled with reactor jacket set to −17° C. 105.8 g of chiral catalyst was charged, followed by 280 g of hydroxylamine and 47 g of deionized water.

When internal temperature has reached −16° C., dosing is started of overall 1997 g of compound of formula II, in 12 portions of about 167 g over 1 hour. The highest internal temperature reached during addition was around −14° C. After the last portion of of compound of formula II has been added, the reaction mass was continued to stir for 2 hours. The reaction mixture was sampled to confirm full conversion. After full conversion, 76 mL 32% HCl aqueous solution was added over 15 min to achieve a pH ˜5.5. The reaction mixture was warmed to room temperature (25° C.) and the pH was again adjusted to finally pH ˜5.0 with additional 5 ml 32% HCl aqueous solution. A sample was taken for analysis (determination of chemical yield and isomer ratio).

The isomer ratio between the isomers A, B, C and D of the compound of formula I, and the chemical yield are gathered in Table 1. The isomers A, B, C and D are defined as follows: A is the isomer (5S,4R); B is the isomer (5S,4S); C is the isomer (SR,4R); and D is the isomer (5R,4S).

TABLE 1

| | | Enantiomeric excess | |
|---|---|---|---|
| | Isomer ratio | (ee) (A + B − C − | Chemical |
| | A:B:C:D % | D) % | yield % |
| Ex. 1 | 95.1:1.5:3.4:0 | 93.2 | 98.2 |
| Ex. 2 | 92.8:2.1:5.0:0.1 | 89.8 | 94.6 |

The results in Table 1 clearly show that the present invention provides an increased enantioselectivity of the desired isomer A (5S,4R) as well as of the enantiomeric excess (ee), while guaranteeing a very good chemical yield.

1. A process for the preparation of a compound of formula I or an enriched composition comprising a compound of formula I 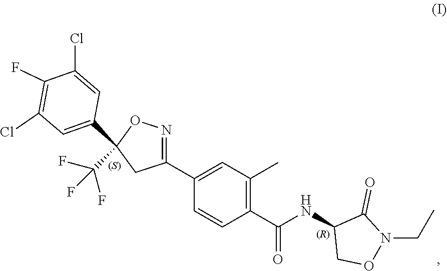
by reacting a compound of formula II 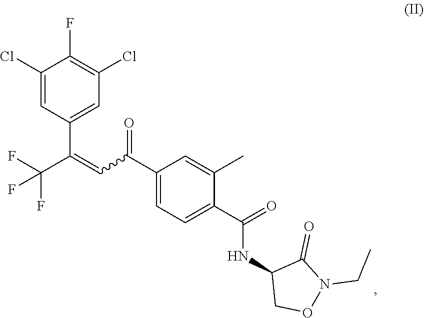 [SMILES: CCN1OC[C@@H](NC(=O)c2ccc(C(=O)C=C(c3cc(Cl)c(F)c(Cl)c3)C(F)(F)F)cc2C)C1=O]
with hydroxylamine or its salt, a base, a chiral catalyst, and an organic solvent, wherein said base is an anion exchange resin.

2. A process according to claim 1, characterized in that the resin is a OH anion exchange resin.

3. A process according to claim 1, characterized in that the anion exchange resin comprise quaternary ammonium functional group.

4. A process according to claim 1, characterized in that the matrix of the anion exchange resin comprises a copolymer of styrene-divinylbenzene.

5. A process according to claim 1, characterized in that the amount of exchangeable anions is from 0.01 to 10 molar equivalents, preferably from 0.05 to 5 molar equivalents, preferably from 0.05 to 1.5 molar equivalents, and more preferably from 0.05 to 0.2 molar equivalents.

6. A process according to claim 1, characterized in that the amount of the organic solvent is from 1 to 200 molar equivalents, and preferably from 10 to 100 molar equivalents.

7. A process according to claim 1, characterized in that it further comprises water.

8. A process according to claim 7, characterized in that the weight ratio of organic solvent: water is from 200:1 to 1:1, and preferably from 100:1 to 5:1.

9. A process according to claim 1, characterized in that the amount of hydroxylamine or its salts can be from 0.5 to 10 molar equivalents, preferably from 0.5 to 5 molar equivalents, and more preferably from 1.0 to 1.5 molar equivalents.

10. A process according to claim 1, characterized in that the amount of the chiral catalyst is from 0.001 to 1.0 molar equivalents, and preferably from 0.01 to 0.5 molar equivalents.

11. A process according to claim 1, characterized in that it comprises, after yielding the compound of formula I, a separation step to remove the anion exchange resin.

12. A process according to claim 1, characterized in that the enriched composition comprises the compound of formula I (5S,4R) and at least one of the isomers of the compound of formula I selected among isomer (5S,4S), isomer (5R,4R), isomer (5R,4S), and any combinations thereof.

13. Use of an anion exchange resin in a process for preparing an isoxazoline group from the cyclisation of a chalcone group.

14. A compound of formula I or an enriched composition comprising a compound of formula I, obtained by a process according to claim 1.
